(19) United States
(12) Patent Application Publication
(10) Pub. No.: US 2022/0082192 A1
(43) Pub. Date: Mar. 17, 2022

(54) FLEXIBLE HOSE WTIH CLOSABLE CUFF, MANUFACTURING METHOD THEREFOR, AND DEVICES CONTAINING

(71) Applicant: VINCENT MEDICAL (DONGGUAN) TECHNOLOGY CO., LTD, Dongguan, Guangdong (CN)

(72) Inventors: Kwok Fu FU, Tangxia, Dongguan, Guangdong (CN); Ming Fai KOH, Tangxia, Dongguan, Guangdong (CN); Ching Sau CHU, Tangxia, Dongguan, Guangdong (CN); Chang Bin MAO, Tangxia, Dongguan, Guangdong (CN); Run Xing LI, Tangxia, Dongguan, Guangdong (CN); Wei Feng HU, Tangxia, Dongguan, Guangdong (CN)

(21) Appl. No.: 17/299,875

(22) PCT Filed: Jan. 30, 2019
(86) PCT No.: PCT/CN2019/073889

§ 371 Date: Jun. 4, 2021

(51) Int. Cl. F16L 25/00; F16L 11/118; F16L 25/01; F16L 33/34; F16L 41/02; A61M 16/08

(52) U.S. Cl. CPC F16L 25/0054; F16L 11/1185; F16L 25/01; A61M 2207/00; F16L 41/021; A61M 16/0816; F16L 33/34

(57) ABSTRACT

A flexible hose has a length of hollow tubing and a closed cuff. A method for manufacturing a flexible hose, has the steps of providing a length of flexible tubing, providing a closable cuff, aligning the closable cuff with the hollow tubing at a closed cuff location, and affixing the closable cuff to the length of hollow tubing by closing the closable cuff around the length of hollow tubing at the closed cuff location, thereby forming a flexible hose having a closed cuff. The method does not include the step of screwing the closable cuff onto the length of hollow tubing. Devices may also contain such a flexible hose.

### FIELD OF THE INVENTION

The present invention relates to the field of flexible hoses, the manufacturing thereof, and devices and systems therefor. More specifically, the present invention relates to the field of flexible hoses having a cuff at an end thereof, the manufacturing thereof, and devices and systems therefor.

### BACKGROUND OF THE INVENTION

Flexible hoses having a cuff on one or more ends is known in many industries, such as in devices from the vacuum cleaner industry, the medical device industry, the liquid pumping industry, the car industry, the industrial hair dryer industry, the industrial steam cleaning industry, etc. These flexible hoses may be intended to carry anything from a high pressure stream, an atmospheric pressure stream, a vacuum, etc. as desired. Furthermore, these flexible hoses may be intended to carry a gas, a liquid, and/or a vacuum as desired. Often these tubes are formed of a plastic, typically a thermoplastic, as this may allow the tube to be both flexible and strong. Specific flexible hoses and methods for manufacturing such flexible hoses are known.

However, the very flexibility of the tube itself means that often the tube is unable to form a tight seal with the device to which it is connected, especially if the tube is to be a removable tube. Thus typically a flexible tube contains a hard plastic cuff at one or multiple ends which is then affixed; or removably-affixed, to a connector, which may be, for example, an overmoulded end piece, which then in turn connects to the device. The connector may be moulded, glued, welded, locked, and/or otherwise affixed to the cuff. Generally, flexible hoses are known to break at the point where the flexible plastic connects to a harder plastic which can cause a loss of pressure, leakage, etc.

Therefore in many cases the cuff provides stress reduction around the point at which a harder plastic is affixed to the flexible hose. This distributes stresses caused by movement of the flexible hose over a greater flexible hose surface area which in turn reduces the chance of flexible tube fracture or breakage at any specific point. The cuff is typically formed of a harder plastic which deforms less than the flexible hose material and thus may affix; or removably-affix, to a connector; or an overmoulded end piece, to allow a secure and tight seal. This in turn requires that the flexible hose protrude from both ends of the cuff, so that at the terminal end, the connector, or the overmoulded end piece, may be bonded to the flexible hose so as to ensure a tight seal. The connector; or the overmoulded end piece, also may connect to the device so as to form a secure and tight seal therewith as well. See for example, U.S. Pat. No. 9,784,387 B2 to Kaye, et al. published on Oct. 10, 2017, and assigned to Plastiflex Group.

More specifically, the medical industry employs flexible hoses in, for example, CPAP (continuous positive airway pressure) systems, oxygen enhancement systems, humidified breathing circuits, heated breathing circuits, ventilation breathing system, anaesthesia breathing systems, surgical insufflation systems, respiratory humidifier systems, etc. These flexible hoses often have a helical external rib which is complementary to an inner threaded screw on the inside of the cuff. In some cases, the cuff may have the combination of an inner threaded screw and a non-threaded cuff portion. The helical external rib may be formed of the same material as the flexible hose, or another material and often serves to improve the rigidity and/or durability of the flexible hose. Furthermore, the helical external rib may contain therein one or more wires therein to, for example, conduct electricity, serve as a sensor, provide control signals, etc. The threaded cuff is then typically affixed by screwing it onto the flexible hose. In some cases, an adhesive is applied to the end of the flexible hose and/or the edge of the cuff prior to screwing it on to the flexible hose. The adhesive then bonds the flexible hose to the cuff.

However, it has been found that the need for the terminal end of the flexible hose to protrude beyond the cuff so as to bond with the connector; or the overmoulded end piece, requires excess flexible hose material. In addition, when an adhesive is applied to the end of the flexible hose and/or the edge of the cuff prior to screwing it on to the flexible hose, it has been found that this may result in the adhesive being applied unevenly between the cuff and the flexible hose, leading to an uneven seal or even an incomplete seal as the adhesive.

Accordingly, it has been found that there remains a need for a flexible hose and associated manufacturing method for providing a more secure and uniform bond between the flexible hose and the cuff. In addition, there exists a need to reduce the amount of flexible hose material used to create a flexible hose.

### SUMMARY OF THE INVENTION

An embodiment of the present invention relates to a flexible hose having a length of hollow tubing and a closed cuff. Another embodiment of the present invention relates to a method for manufacturing a flexible hose, having the steps of providing a length of flexible tubing, providing a closable cuff, aligning the closable cuff with the hollow tubing at a closed cuff location, and affixing the closable cuff to the length of hollow tubing by closing the closable cuff around the length of hollow tubing at the closed cuff location, thereby forming a flexible hose having a closed cuff. The method does not include the step of screwing the closable cuff onto the length of hollow tubing.

A device may also contain the flexible hose described herein.

Without intending to be limited by theory, it is believed that the present invention herein provides improved flexible tubing as well as an improved manufacturing method. It is believed that the flexible hose of the present invention may possess a more uniform and more accurately-applied bond between the flexible hose and the closed cuff. It is also believed that the present method herein may more accurately position and/or affix the closable cuff to the hollow tubing. It is also believed that the present manufacturing method may result in reduced wasted flexible hose material. It is also believed that the present invention may significantly reduce the amount of manufacturing time and/or complexity.

### BRIEF DESCRIPTION OF THE DRAWINGS

FIG. 1 shows a partially-cut away side view of an embodiment of a flexible hose and a closable cuff of the present invention;

FIG. 2 shows a side view of an embodiment of the hollow tubing and the closable cuff of FIG. 1, as they are being affixed to each other;

FIG. 3 shows a perspective view of a closable cuff having a continuous helical thread on the closable cuff inner surface which is formed of multiple cuff portions

FIG. 4 shows a partial side view of an embodiment of the flexible hose of the present invention having hollow tubing which has a closed cuff at the terminal end thereof;

FIG. 5 shows a partial side-view of an embodiment of the present invention having a flexible hose with a length of hollow tubing which has a closed cuff at each terminal end; and

FIG. 6 shows a side view of a cuff portion of a multiple-piece cuff having a closable cuff inner surface and a closable cuff outer surface.

The figures herein are for illustrative purposes only and are not necessarily drawn to scale.

### DESCRIPTION OF THE PREFERRED EMBODIMENTS

Unless otherwise specifically provided, all tests herein are conducted at standard conditions which include a room and testing temperature of 25° C. and sea level (1 atm.) pressure, and all measurements are made in metric units. Furthermore, all percentages, ratios, etc. herein are by weight, unless specifically indicated otherwise.

As used herein, the term “comprising” as used in the claims should not be interpreted as being exclusively restricted to the claim elements listed thereafter, and this term indicates that other claim elements, steps, etc. may also be included without deviating from the scope of the invention. This term is interpreted to specify the presence of the listed features, elements, steps, components, etc. referred to, but does not preclude the presence of one or more other features, elements, steps, components, etc. or groups thereof.

As used herein, any terms such as first, second, third, and the like in the description and the claims are merely used for distinguishing between similar elements and are not necessarily for describing a requires sequential or chronological order. One skilled in the art further understands that these terms may be interchangeable under the appropriate circumstances.

As used herein, the term “closed cuff” and its variations such as “closable cuff” indicates a cuff which is either open or has a larger diameter which is then aligned with and/or positioned on the hollow tubing and then closed around the tubing. A closable cuff is therefore different from a cuff that is either slid or screwed onto the hollow tubing because the action of affixing the closable cuff is different. Specifically, the closable cuff changes diameter and/or shape when being affixed to the hollow tubing to form the closed cuff. In contrast, a cuff which is slid or screwed onto the hollow tubing has a diameter which remains significantly the same at all times. In an embodiment herein, the closed cuff/closable cuff is not screwed onto the hollow tubing; or is not slid onto the hollow tubing, screwed onto the hollow tubing, or a combination thereof. As used herein, the terms “closed cuff” and “closable cuff” and their variations are intended to be virtually interchangeable, but one or the other is merely chosen to indicate the status of the closable/closed cuff at that specific point in time.

The present invention relates to a flexible hose having a length of hollow tubing, and a closed cuff. The flexible hose herein may be manufactured according to many different methods known in the art such as, for example, extrusion, injection moulding, vacuum forming, and a combination thereof; or extrusion, and a combination thereof. The length of the hollow tubing may be any length desired, from a few centimeters to many hundreds of meters or more and anywhere in-between. The flexible hose may be either manufactured to a specific length such as via injection moulding or vacuum forming, or may be manufactured in a continuous process, such as during an extrusion process, and cut to a desired length either immediately or at a later point in the manufacturing process.

The hollow tubing and/or the flexible hose may contain at least a single lumen; or a plurality of lumens as desired; or from about 1 to about 5 lumens; or from about 1 to about 3 lumens; or 1 lumen; or 2 lumens; or 3 lumens. If a plurality of lumens are present, then the multiple lumens may be concentric, or non-concentric as desired. Generally, hollow tubing and methods for making such are well-known in the art. See for example, U.S. Pat. No. 6,431,218 B1 to Woelful and Britten, published on Aug. 13, 2002 and assigned to Vital Signs, Inc. of Totawa, N.J., USA.

In an embodiment herein, the hollow tubing may contain a plastic, a resin, a metal, a carbon fiber, and a combination thereof; or plastic, a resin, a metal, and a combination thereof; or a plastic, a metal, and a combination hereof; or a thermoplastic, a wire, and a combination thereof; or polyethylene, polypropylene, polyolefin, polystyrene, polyvinylchloride, metallocene polyethylene, a wire, polyolefin elastomer, and a combination thereof. In an embodiment herein, the hollow tubing contains a material that is approved according to ISO 10993 for use in medical applications and products.

In an embodiment herein, the hollow tubing contains a hollow tubing outer surface and a hollow tubing inner surface opposite the hollow tubing outer surface. Typically the gas, vacuum, liquid, etc. to be transported or conveyed by the hollow tubing will be inside of the hollow tubing inner surface.

In an embodiment herein, the hollow tubing outer surface contains a helical rib thereupon. In an embodiment herein, the helical rib is produced by extrusion; or produced by extrusion at the same time the hollow tubing is produced. See, for example, U.S. Pat. No. 4,490,575 to Kutnyak, assigned to Automation Industries, Inc. of Greenwich, Conn., USA, published on Dec. 25, 1984. In another embodiment, the helical rib is produced separately and added to the hollow tubing outer surface after the initial manufacture of the hollow tubing. In an embodiment herein, the helical rib contains a wire; or a plurality of wires. In an embodiment herein, the helical rib contains a wire; or a plurality of wires, therein. In an embodiment herein, the helical rib contains a sensor. In an embodiment herein, the helical rib contains a sensor, therein.

In an embodiment herein, the helical rib may be formed of a plastic, a resin, a metal, a carbon fiber, and a combination thereof; or plastic, a resin, a metal, and a combination thereof; or a plastic, a metal, and a combination hereof; or a thermoplastic, a metal, and a combination thereof; or polyethylene, polypropylene, polyolefin, polystyrene, polyvinylchloride, metallocene polyethylene, a metal, and a combination thereof; or polypropylene, a wire, and a combination thereof. In an embodiment herein, the helical rib contains a material that is approved according to ISO 10993 for use in medical applications and products.

Without intending to be limited by theory, it is believed that the helical rib may enhance the structural stability, crush resistance (a.k.a., hub strength, hoop strength, etc.), and/or other physical properties of the flexible hose and/or the hollow tubing. Without intending to be limited by theory, it is also believed that the helical rib may also protect the wire and/or sensor therein from damage.

The closed cuff herein indicates a cuff that is not screwed onto, and/or not slid onto the hollow tubing to affix the cuff. In contrast, the closed cuff changes shape and closes about the hollow tubing. The closable cuff may be a multiple-piece cuff, a hinged cuff, and a combination thereof; or a multiple-piece cuff, a hinged cuff, and a combination thereof. Thus, in an embodiment herein the closed/closable cuff is a multiple-piece cuff that contains a plurality of separate pieces; or from about 2 pieces to about 6 pieces; or from about 2 pieces to about 4 pieces, that are affixed together around the hollow tubing so as to form the closed cuff.

In an embodiment herein the closed cuff is a hinged cuff formed of a single piece having two or more cuff portions connected via a hinge/hinges. In order to affix the hinged cuff to the hollow tubing, the cuff portions are brought together, thereby bending and/or rotating the hinge or hinges so as to form a closed cuff.

In an embodiment herein, the closed cuff contains a closed cuff inner surface and a closed cuff outer surface opposite the closed cuff inner surface. The closed cuff inner surface contacts and/or is proximal to the hollow tubing; or the hollow tubing outer surface. The closed cuff outer surface faces away from and/or is distal from the hollow tubing.

In an embodiment herein, the closable cuff or closed cuff contains a closure to help affix the closable/closed cuff onto the hollow tubing. The closure may be selected from the group of a snap-fit closure, a post and hole closure, a shrinkable closure, a welded closure, a glued closure, and a combination thereof; or a snap-fit closure, a post and hole closure, and a combination thereof. Without intending to be limited by theory, it is believed that such a closure helps to ensure that the closed cuff does not become unintentionally detached from the hollow tubing.

Typically the length of hollow tubing will have at least one terminal end where the hollow tubing terminates. The closed cuff will typically be located at a closed cuff location which may be at the terminal end. In an embodiment herein, the flexible hose contains a terminal end and a closed cuff affixed to the terminal end. In an embodiment herein, the flexible hose contains a plurality of terminal ends. In an embodiment herein, the flexible hose contains a closed cuff affixed to a plurality of terminal ends; or a closed cuff affixed to each terminal end. In an embodiment herein, the flexible hose contains from about 1 terminal end to about 10 terminal ends; or from about 2 terminal ends to about 5 terminal ends; or from about 2 terminal ends to about 4 terminal ends; or from about 2 terminal ends to about 3 terminal ends. In an embodiment herein, the flexible hose contains a second closed cuff, and the second closed cuff is distal to the closed cuff.

In an embodiment herein, where the hollow tubing contains a helical rib, the closed cuff inner surface contains a helical thread. The helical thread is complimentary to the helical rib on the hollow tubing outer surface. In an embodiment herein, the helical thread is a continuous helical thread. In an embodiment herein, the helical thread is a broken helical thread.

An embodiment herein further contains an overmoulded end piece which is affixed; or permanently-affixed, to the closed cuff. The overmoulded end piece typically connects; or removably-connects to a device such as from the group of an engine, a blower, a medical device, a steam device, a vacuum system, a ventilator, an anaesthesia station, a CPAP machine, a humidifier chamber, a nebulizer, a mask, a tracheal tube, a filter, a suction machine, an oxygen concentrator, an insufflation device, and a combination thereof; or a medical device, a vacuum system, a ventilator, a humidifier, a humidifier water chamber, an insufflation device, and a combination thereof; or a CPAP (continuous positive airway pressure) system, an oxygen enhancement system, a breathing circuit, an anaesthesia system, a surgical insufflation system, a humidifier, a humidifier water chamber, an insufflation device, and a combination thereof; or a breathing circuit, a humidifier water chamber, an insufflation device, and a combination thereof. In an embodiment herein, the breathing circuit is selected from the group of a humidified breathing circuit, a heated breathing circuit, and a combination thereof.

In an embodiment herein, the overmoulded end piece may be permanently affixed to the closed cuff and/or the flexible tubing so as to form a seal; or a tight seal; or a water-tight seal, a vacuum-tight seal, an air-tight seal, or a combination thereof.

In an embodiment herein, the hollow tubing does not extend past the terminal end of the closed cuff, and in fact terminates within the closed cuff. In such an embodiment, the overmoulded end piece may be permanently affixed to the closed cuff so as to form a seal; or a tight seal; or a water-tight seal, a vacuum-tight seal, an air-tight seal, or a combination thereof.

In an embodiment herein, the closed cuff contains an electrical interface. In an embodiment herein, the connector; or the overmoulded end piece; or the port, contains an electrical interface. The electrical interface in the closed cuff may, for example, serve as a link between the wire, sensor, etc. in the helical rib, and the electrical interface in connector; or the overmoulded end piece. In an embodiment herein, the electrical connector may, for example, serve as a power source for the wires, especially if the wires serve as heating wires.

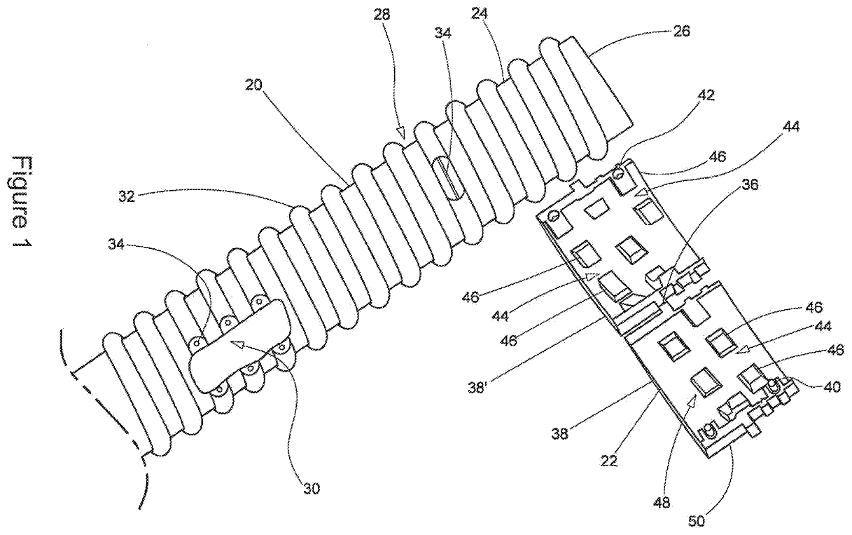

Turning to the Figures, FIG. 1 shows a partially-cut away side view of an embodiment of a flexible hose, 20, and a closable cuff, 22, of the present invention. The flexible hose, 20, contains a length of hollow tubing, 24, which terminates in a terminal end, 26. The hollow tubing, 24, in FIG. 1 contains the hollow tubing outer surface, 28, as well as the hollow tubing inner surface, 30, opposite the hollow tubing outer surface, 28, and visible through the cut-away view. Also available in the partial cut-away view is a helical rib, 32, which winds around the hollow tubing outer surface, 28. Also visible in FIG. 1 is a wire, 34, which may also serve as a sensor. However, as discussed herein, multiple wires, sensors, etc. may be present in the helical rib, or elsewhere in the hollow tubing.

The closable cuff, 22, contains a hinge, 36, which connects two cuff portions, 38 and 38′. The hinge, 36, bends when the cuff portions, 38 and 38′, are closed to affix the closable cuff, 22, to the hollow tubing, 24. The closable cuff, 22, also contains a post, 40, and a corresponding hole, 42. When the cuff portions, 38 and 38′, are closed to affix the closable cuff, 22, to the hollow tubing, 24. The post, 40, enters the hole, 42, to help align and/or keep the cuff portions, 38 and 38′, together. The cuff portions, 38 and 38′, also contain a broken helical thread, 44, formed of different blocks, 46, that are moulded into the cuff portions, 38 and 38′, themselves. The broken helical thread, 44, is complementary to the helical rib, 32, when the closable cuff, 22, is affixed to and closed around the hollow tubing, 24.

The closable cuff, also contains a closable cuff inner surface, 48, and a closable cuff outer surface, 50, opposite to the closable cuff inner surface, 48. The broken helical thread, 44, and the blocks, 46, are all located in the closable cuff inner surface, 48.

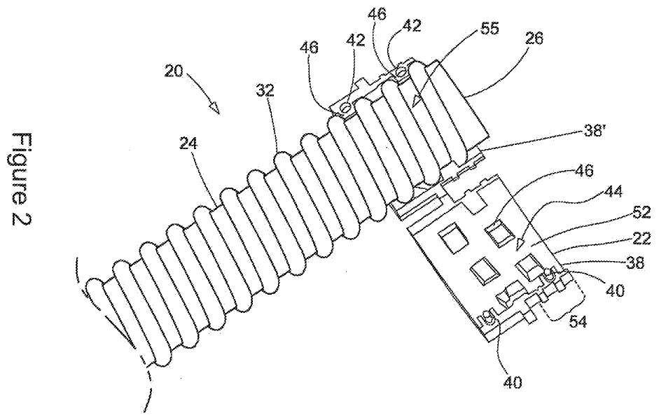

Turning to FIG. 2, this figure shows a side view of an embodiment of the hollow tubing, 24, and the closable cuff, 22, of FIG. 1, as they are being affixed to each other. Thus, in an embodiment of the method herein, an adhesive, 52, is provided and applied to the cuff portion, 38 and 38′, at a specific adhesive location, 54. The adhesive, 52, may be specifically provided in sufficient quantity and specifically at the desired adhesive location, 54, so as to form a seal; or a tight seal; or an air-tight seal, a water-tight seal, or a combination thereof, between the hollow tubing, 24, and the closable cuff, 22, and/or the cuff portions, 38 and 38′.

Without intending to be limited by theory, it is believed that since the cuff portions, 38 and 38′, in FIGS. 1 and 2 are open when the adhesive is applied thereto, it is easy and practical for the manufacturer to provide enough adhesive, and at the specific, desired adhesive location(s) so as to ensure that a seal is formed each and every time when the closable cuff is affixed to the hollow tubing. In contrast, with previous manufacturing methods, when an adhesive is applied to the terminal end of the hollow tubing and then the normal (e.g., already-closed) cuff is screwed onto the hollow tubing, the completeness of the seal in each case will be different as the adhesive, hollow tubing, and normal cuff will interact differently each time. When screwing on a normal cuff to the hollow tubing, it is therefore impossible to ensure that a seal, or a tight seal; or a water-tight seal; or an airtight seal, is formed each and every time. Furthermore, if extremely viscous adhesives, such as silicone gels, are employed, it may be difficult to screw a normal cuff onto the hollow tubing, which is quite flexible, as the required torque may deform the hollow tubing. However, it is believed that the method herein avoids the inconsistency and incomplete seal problems caused when applying the adhesive and then screwing the cuff onto the hollow tubing. Accordingly, it is believed that while the terminal end, 26, protrudes from the closed cuff, 22, in FIG. 2, such a feature may not be needed at all, to ensure an acceptable seal.

In FIG. 2 it is also evident that the broken helical thread, 44, is complementary to the helical rib, 32, as it can be seen that the blocks, 46, are complementary to the helical rib, 32. The post, 40, would then fit into the corresponding hole, 42, when the cuff portions 38, and 38′, are closed to affix the closable cuff, 22, to the hollow tubing, 24. The adhesive, 52, may also serve to keep the cuff portions, 38 and 38′, affixed to each other as well as to the hollow tubing, 24.

The closed cuff, 22, is aligned with the hollow tubing, 24, at a closed cuff location, 55, which indicates the intended location on the hollow tubing, 24, where the manufacturer wishes to affix the closed cuff, 22.

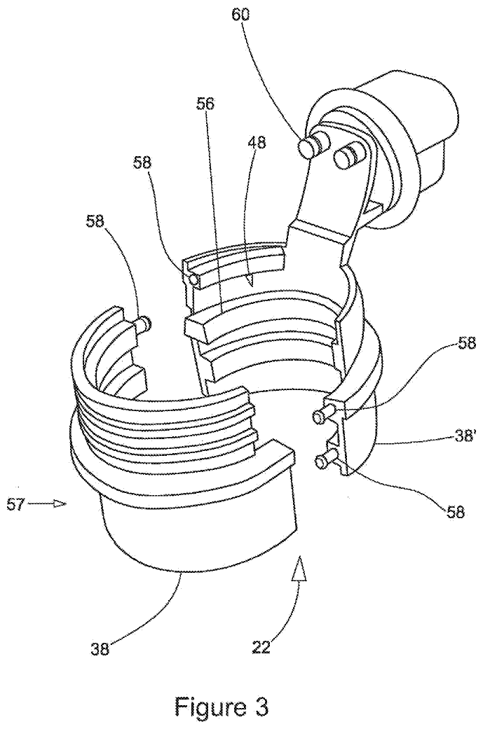

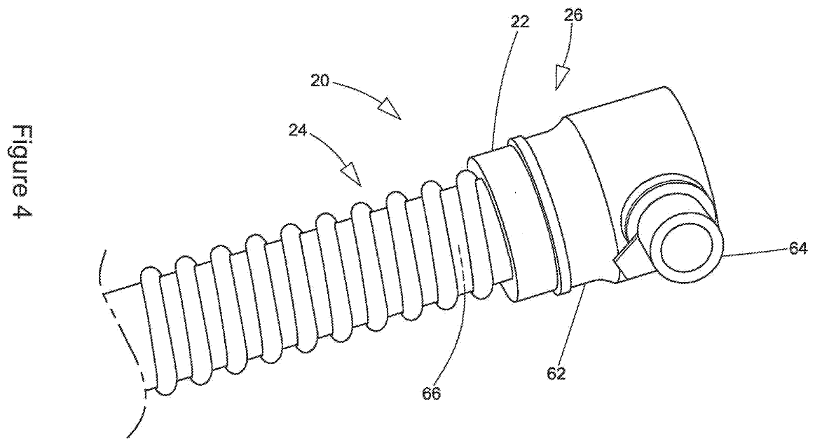

FIG. 3 shows a perspective view of a closable cuff, 22, having a continuous helical thread, 56, on the closable cuff inner surface, 48, which is formed of multiple cuff portions, 38 and 38′; thus this is a multiple-piece cuff, 57. The cuff portions, 38 and 38′, also contain complementary portions of a snap-fit closure, 58, on each separate cuff portion, 38 and 38′. The post of the snap-fit closure, 58, fits into the corresponding hole of the snap-fit closure, 58, to close the cuff portions, 38 and 38′, and keep them together. The closable cuff, 22, also has an electrical interface, 60, which may be directly- or indirectly-connected to, for example, the wire (see FIG. 1 at 34) and/or the sensor in the helical rib (see FIG. 1 at 32), and/or an electrical interface, 60, in an overmoulded end piece (see FIG. 4 at 62). As with the hinged closable cuff in FIGS. 1-2, the multiple-piece closable cuff, 22, also allows the manufacturer to provide enough adhesive, and at the specific, desired adhesive location(s) so as to ensure that a the desired seal is more consistently-formed when the closable cuff is affixed to the hollow tubing.

FIG. 4 shows a partial side view of an embodiment of the flexible hose, 20, of the present invention having hollow tubing, 24, which has a closed cuff, 22, at the terminal end, 26, thereof. The closed cuff, 22, is further affixed to an overmoulded end piece, 62. The overmoulded end piece, 62, may be formed and/or moulded directly onto the closed cuff, 22, or may be otherwise affixed thereupon, by employing, for example, adhesives, ultrasonic welding, etc. The overmoulded end piece may be also directly-affixed to, for example, the terminal end, 26, of the hollow tubing, 24, especially if the terminal end, 26, protrudes from the closed cuff, 24, as seen in FIG. 2. The overmoulded end piece, 62, also contains a port, 64, that is operatively-connected to the interior of the hollow tubing. The port, 64, may further directly (or operatively-) connect to, for example, an additional device such as a thermostat, a humidifier, a ventilator, an insufflation device, etc. In an embodiment herein, the port, 64, allows a sensor to enter into the lumen, 66, of the flexible hose, 20.

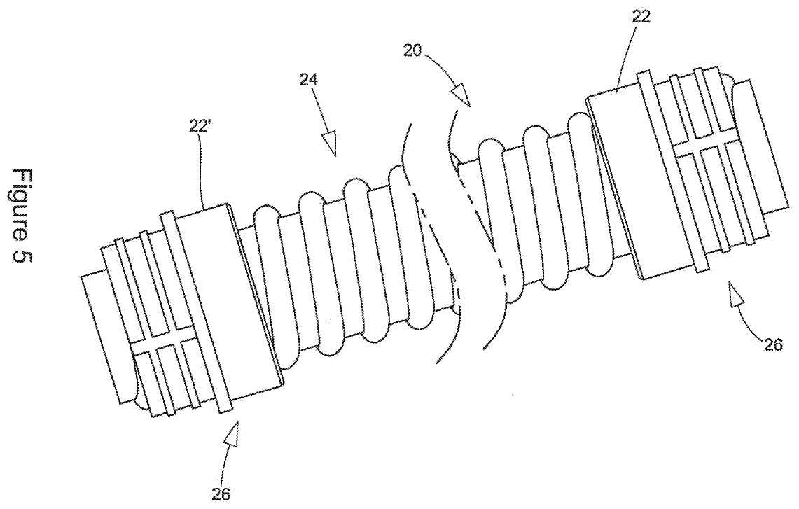

FIG. 5 shows a partial side-view of an embodiment of the present invention having a flexible hose, 20, with a length of hollow tubing, 24, which has a closed cuff, 22, at each terminal end, 26. In this embodiment, the second closed cuff, 22′, is distal from the closed cuff, 22.

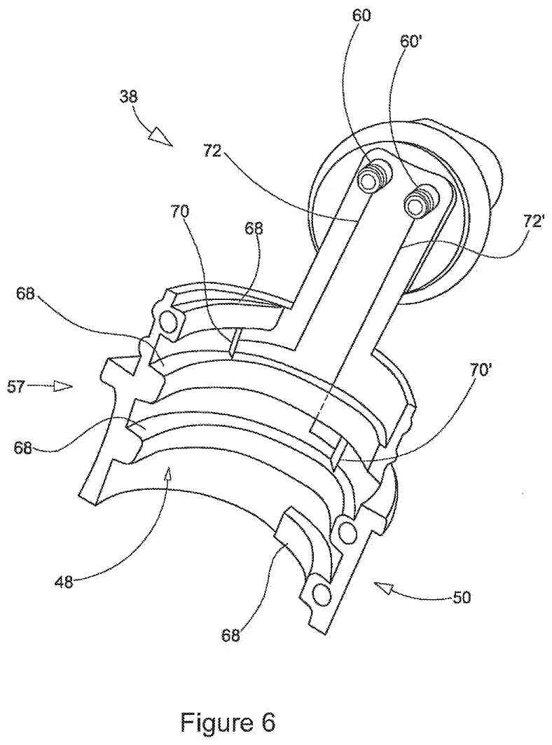

FIG. 6 shows a side view of a cuff portion, 38, of a multiple-piece cuff, 57, having a closable cuff inner surface, 48, and a closable cuff outer surface, 50. The cuff portion, 38, also contains a helical thread, 68, which is complementary to the helical rib (see, e.g., FIG. 1 at 32). In between the helical thread, 68, is located a plurality of blades, 70. When the closable cuff, 38, is affixed to the terminal end (see FIG. 1 at 26) of the hollow tubing (see FIG. 1 at 24) and compressed against the hollow tubing (see FIG. 1 at 24) with sufficient force, the blade, 70, cuts through the helical rib (see FIG. 1 at 32) to contact the wire (see FIG. 1 at 34) thereby creating an electrical connection that operatively-connects the wire (see FIG. 1 at 34) with the blade, 70.

The blade, 70, is connected to a blade wire, 72, which forms an electrical connection between the blade, 70, and the electrical interface, 60. The electrical interface, 60, then may operatively-connect, either directly or indirectly to the overmoulded end piece (see FIG. 4 at 62), and/or the port, 64. In an embodiment herein, the electrical interface, the overmoulded end piece, and/or the port may be further operatively-connected to a machine, or other device.

The blade wire, 72, may be either on the closable cuff inner surface, 48, the closable cuff outer surface, 50, or may be embedded within the material of the closable cuff, 32, as desired. Without intending to be limited by theory, it is believed that an embodiment such as that of FIG. 6 may allow significantly faster manufacturing, as it removes the need for a person to separately solder an electrical connection between the wire in the helical rib and the electrical interface on the closable cuff.

In an embodiment herein, the closable cuff contains at least 1 blade; or a plurality of blades; or from about 2 blades to about 4 blades; or from about 2 blades to about 3 blades; or about 2 blades. In an embodiment herein where the closable cuff contains a plurality of blades, then the flexible hose contains an equal number of separate helical ribs, each containing at least one wire therein. If there are a plurality of wires, then in an embodiment herein, each wire is electrically distinct from the other wire(s).

Method of Manufacturing

In an embodiment herein, the present invention relates to a method for manufacturing a flexible hose having the steps of providing a length of hollow tubing, providing closable cuff, aligning the closable cuff with the hollow tubing at a closed cuff location, and affixing the closed cuff to the hollow tubing. The affixing step may be achieved by closing the closable cuff around the length of hollow tubing at the closable cuff location to form a length of tubing having a closed cuff. However, in doing so the method herein does not include the step of screwing the closable (or closed) cuff onto the length of hollow tubing, as this may defeat the purpose of the invention, which is to avoid the need for screwing the cuff onto the hollow tubing.

In an embodiment herein, the method further includes the step of applying an adhesive to an adhesive location prior to the affixing step where the closable cuff is affixed to the length of hollow tubing. In an embodiment herein, the adhesive location is selected from the group of a location on the hollow tubing, a location on the closable cuff, and a combination thereof. Without intending to be limited by theory, it is believed that the method of affixing the closable cuff herein to the hollow tubing avoids potential issues, such as the smearing, imprecise distribution of the adhesive, and incomplete seal formation that may occur when the adhesive is first applied to the terminal end of the hollow tubing and then the cuff is screwed onto the hollow tubing. Alternatively, it is believed that the present method also avoids the similar problems which may occur when an adhesive is first applied to a portion of a normal, ring-like cuff and then the cuff is screwed onto the hollow tubing. As the method allows precise application of an adhesive to an application location, and the high likelihood that the adhesive will not be significantly moved from that adhesive location, it is believed that the present method herein provides a more consistent adhesion, a more consistent seal, improved quality control, a reduction in the amount of adhesive required to form the desired seal, the use of adhesives that are unacceptable in other assembly processes, and/or simplifies the assembly process, especially the cuff assembly process.

In an embodiment herein, the flexible hose contains more than 1 closable cuff.

Therefore an embodiment of the method of manufacture herein also contains the steps of providing a second closable cuff, aligning the second closable cuff with the hollow tubing at a second closable cuff location distal to the closed cuff location, and affixing the second closable cuff to the length of hollow tubing at the second closable cuff location. The affixing of the second closable cuff to the hollow tubing may be achieved by closing the second closable cuff around the length of hollow tubing to form a length of hollow tubing comprising a second closed cuff. As with the (first) closed cuff described above, the affixing step specifically does not comprise screwing the second closable cuff onto the length of hollow tubing.

In an embodiment of the present invention, the method herein further contains the step of affixing a connector; or an overmoulded end piece, to the closed cuff. The connector; or the overmoulded end piece, may be affixed to the closed cuff by, for example, moulding the connector; or the overmoulded end piece, directly onto the closed cuff, by employing an adhesive between the connector; or the overmoulded end piece, and the closed cuff, by press-fitting the connector; or the overmoulded end piece, to the closed cuff, by ultrasonically sealing the connector; or the overmoulded end piece, to the closed cuff, and a combination thereof; or moulding the overmoulded end piece directly onto the closed cuff.

One skilled in the art understands that the hollow tubing may be formed by, for example, the methods already described herein, such as simultaneously forming a hollow tubing via extrusion, and forming a moulded rib via extrusion. See, for example, U.S. Pat. No. 4,490,575 to Kutnyak, assigned to Automation Industries, Inc., published on Dec. 25, 1984. Typically, the moulded rib is formed integral with the hollow tubing; or on the hollow tubing outer surface, and may contain, for example, a wire; or a plurality of wires, and/or a sensor therein.

### Devices

The flexible hose herein may be connected to virtually any type of device which moves a gas, water, or vacuum from one place to another. Non-limiting examples of such devices include those known in the vacuum cleaner industry, medical device industry, the liquid pumping industry, the car industry, the industrial hair dryer industry, the industrial steam cleaning industry, etc. More specifically, in an embodiment herein, the device herein may be, for example, a respirator, a vacuum cleaner, a clothes steamer, a ventilator, a humidifier, a humidifier water chamber, a nebulizer, a mask, a tracheal tube, an insufflation device, and a combination thereof.

In an embodiment herein, the method of manufacturing herein further includes the steps of providing a closable cuff having a blade, aligning the blade with a helical rib, and cutting the helical rib with the blade such that the blade contacts the wire. It is believed that the present invention may remove the need to separately hand-solder the wire in the helical rib to the electrical interface. It is also believed that this manufacturing process improvement may significantly reduce manufacturing cost, time, and may also reduce human variability in the production process.

It should be understood that the above only illustrates and describes examples whereby the present invention may be carried out, and that modifications and/or alterations may be made thereto without departing from the spirit of the invention.

It should also be understood that certain features of the invention, which are, for clarity, described in the context of separate embodiments, may also be provided in combination in a single embodiment. Conversely, various features of the invention which are, for brevity, described in the context of a single embodiment, may also be provided separately, or in any suitable subcombination.

While not specifically admitting to their relevance and/or availability as prior art, all references cited herein are incorporated by reference in their entirety unless otherwise specifically noted herein.

The invention claimed is:

1. A flexible hose comprising:
A) a length of hollow tubing; and
B) a closed cuff.

2. The flexible hose according to claim 1, wherein the hollow tubing comprises a plastic.

3. The flexible hose according to claim 1, wherein the hollow tubing comprises:
A) a hollow tubing inner surface; and
B) a hollow tubing outer surface opposite the hollow tubing inner surface, wherein the hollow tubing outer surface comprises a helical rib.

4. The flexible hose according to claim 3, wherein the closed cuff comprises a closed cuff inner surface and a closed cuff outer surface opposite the closed cuff inner surface, and wherein the closed cuff inner surface comprises a helical thread; or wherein the helical thread is a continuous helical thread; or
wherein the helical thread is a broken helical thread, and wherein the helical thread is complementary to the helical rib.

5. The flexible hose according to claim 3, wherein the helical rib further comprises a wire therein.

6. The flexible hose according to claim 1, wherein the closed cuff is selected from the group consisting of a multiple-piece cuff, a hinged cuff, and a combination thereof.

7. The flexible hose according to claim 1, wherein the closed cuff comprises a closure selected from the group consisting of a snap-fit closure, a post and hole closure, and a combination thereof; or a snap-fit closure, a post and hole closure, and a combination thereof.

8. The flexible hose according to claim 1, further comprising a second closed cuff, wherein the second closed cuff is distal from the closed cuff.

9. The flexible hose according to claim 1, further comprising a connector; or an overmoulded end piece, affixed to the closed cuff.

10. The flexible hose according to claim 1, wherein the closed cuff further comprises a blade.

11. A method for manufacturing a flexible hose comprising the steps of:
A) providing a length of hollow tubing;
B) providing a closable cuff;
C) aligning the closable cuff with the hollow tubing at a closed cuff location; and
D) affixing the closable cuff to the length of hollow tubing by closing the closable cuff around the length of hollow tubing at the closed cuff location to form a length of hollow tubing comprising a closed cuff, wherein the method does not comprise the step of screwing the closable cuff onto the length of hollow tubing.

12. The method for manufacturing a flexible hose according to claim 11, further comprising the step of applying an adhesive to an adhesive location selected from the group consisting of a location on the length of hollow tubing, a location on the closable cuff, and a combination thereof, the adhesive applying step occurring prior to the affixing step.

13. The method for manufacturing a flexible hose according to claim 11, further comprising the steps of:
A) providing a second closable cuff;
B) aligning the second closable cuff with the hollow tubing at a second closable cuff location distal from the closed cuff location; and
C) affixing the second closable cuff to the length of hollow tubing at the second closable cuff location by closing the second closable cuff around the length of hollow tubing to form a length of hollow tubing comprising a second closed cuff, wherein the affixing step does not comprise screwing the second closable cuff onto the length of hollow tubing.

14. The method for manufacturing a flexible hose according to claim 11, further comprising the step of affixing a connector; or an overmoulded end piece, to the closed cuff.

15. The method for manufacturing a flexible hose according to claim 14, further comprising the step of affixing the connector; or the overmoulded end piece, to the terminal end.

16. The method for manufacturing a flexible hose according to claim 11, further comprising the steps of forming a hollow tubing via extrusion, and simultaneously forming a moulded rib via extrusion, where the moulded rib is formed integral with the hollow tubing.

17. The method for manufacturing a flexible hose according to claim 11, further comprising the steps of providing a closable cuff having a blade, aligning the blade with a helical rib, and cutting the helical rib with the blade such that the blade contacts the wire.

18. A device comprising the flexible hose according to claim 1.

19. The device according to claim 18 wherein the device is selected from the group consisting of a respirator, a vacuum cleaner, a clothes steamer, a respiratory humidification device, a ventilation breathing device, an anaesthesia breathing device, a surgical insufflation device and a combination thereof.

20. A device comprising the flexible hose manufactured according to the method of claim 11.
